(19) United States
(12) Patent Application Publication
(10) Pub. No.: US 2009/0269830 A1
(43) Pub. Date: Oct. 29, 2009

(54) Culture System and Method for Propagation of Human Blastocyst-Derived Stem Cells

(75) Inventors: Henrik Semb, Bjarred (SE); Raimund Strehl, Goteborg (SE); Catharina Ellerstrom, Vastra Frolunda (SE)

Correspondence Address: BUCHANAN, INGERSOLL & ROONEY PC, POST OFFICE BOX 1404, ALEXANDRIA, VA, 22313-1404, US

(73) Assignee: Cellartis AB, Goteborg (SE)

(21) Appl. No.: 12/225,201

(22) PCT Filed: Mar. 16, 2007
(86) PCT No.: PCT/EP2007/002346

§ 371 Date: Jan. 27, 2009

Related U.S. Application Data
(60) Provisional application No. 60/783,054, filed on Mar. 17, 2006.
(60) Provisional application No. 60/851,743, filed on Oct. 16, 2006.

(30) Foreign Application Priority Data
Mar. 17, 2006 (DK) PA 2006 00381
Oct. 17, 2006 (DK) PA 2006 01344

(51) Int. Cl. C12N 9/76; C12N 5/08; C12N 9/00; C12N 9/48

(52) U.S. Cl. 435/213; 435/366; 435/371; 435/183; 435/212

(57) ABSTRACT

The present invention relates to a culture system for and a method for propagation of human blastocyst-derived stem cells (hBS cells) upon enzymatic dissociation into a single cell suspension. The culture system for propagation of human blastocyst-derived stem (hBS) cells comprises
i) human feeder cells at a density of at least 50,000 cells/cm²,
ii) one or more dissociation agents for dissociation of hBS cell colonies into a single cell suspension, and
iii) a supportive culture medium,
   which culture system makes it possible to propagate hBS cells by dissociation of hBS cell colonies into a single cell suspension at each consecutive passage for an extended time period, while maintaining the significant characteristics of hBS cells.

### FIELD OF THE INVENTION

The present invention relates to a culture system for and a method for propagation of human blastocyst-derived stem cells (hBS cells) upon enzymatic dissociation into a single cell suspension.

### BACKGROUND OF THE INVENTION

Traditionally human blastocyst-derived stem cells (hBS cells) are maintained on a mEF (mouse embryonic fibroblast) feeder cell layer and are propagated by manual cutting and transfer of individual pieces of colonies [Heins et al, WO03055992, Bresagen]. This traditional culture is very labor intensive and time consuming but is to date the preferred culture method which allows the maintenance of hBS cell lines in a stable, normal state over extended periods of time and is therefore a suitable culture system for smaller-scale maintenance culture. However, there are many technical drawbacks associated with this traditional culture system. For instance it is nearly impossible to quantify how many cells are being transferred in one piece, which in turn has negative effects on reproducibility and standardization. Up-scaling processes based on these traditional culture systems are limited due to low compatibility with automation techniques, such as robots and bioreactors and would instead require a huge amount of man-hours, laboratory space and equipment, such as microscopes. Furthermore, techniques for cell sorting both for terminal analysis and for subculture of sorted cells by e.g. density gradients, FACS (fluorescence automated cell sorting) or magnetic bead sorting as well as cell transfection techniques by e.g. electroporation or viral agents are all preferably performed on dissociated single cells than on pieces or clusters of cells.

Enzymatic dissociation would therefore render propagation of hBS cells more efficient in terms of the time and labor required. Furthermore, dissociation of hBS cells into single cells at passage would allow them to be more precisely quantified and to be submitted to a number of manipulating procedures that will expand the scope of potential uses applications of the hBS cells and furthermore it would facilitate automated propagation procedures.

Several groups have attempted enzymatic dissociation but very few have reported propagation of hBS cells upon dissociation into single cell suspensions at passage and have instead relied on passaging by using e.g. collagenase IV, whereby clusters of certain sizes are obtained [Brimble et al, Bresagen, Sjögren-Jansson et al]. For example, while Sjögren-Jansson reports increased adhesion and survival when avoiding single cell passaging in a feeder-free culture system, Brimble and Bresagen report that they use cluster sizes of around 10-100 cells and further clearly recommend avoiding dissociation to single cells during passaging, as it has negative effects on viability (Human Embryonic Stem Cell Protocols). Still other groups have performed enzymatic dissociation of hBS cells and found it to be necessary to expose the cells to enzymes already during the establishment or during the very early passages of the hBS cell line in order adjust the cells to the enzymes [Cowan et al] whereby only hBS cell lines established according to these more recent enzymatic protocols and not the majority of the today existing hBS cell lines (traditionally established and cultured) would be applicable for enzymatic passaging and potential automated larger scale propagation and expansion. Furthermore, only relatively low passage ratios or split ratios (1:3) have been described when employing enzymatic dissociation into single cells at passage [Cowan et al], which have implied poor possibilities for up-scaling the propagation to larger-scale propagation.

In addition to the above-mentioned technical difficulties relating to enzymatic dissociation into single cells upon passage, problems regarding the stability and the quality of the propagated hBS cell lines have been reported [Draper et al, Buzzard et al, Mitalipova, Enver et al, Andrews et al]. For example, Draper, Buzzard and Mitalipova describe introduction of chromosomal aberrations, such as gain of chromosomes 12 and 17q. Also Cowan clearly indicates genetic instability and therefore suggests regular karyotyping. Enver and Andrews describe transformation towards a culture-adapted or in-vitro adapted cell line, which may start to resemble human embryonal carcinoma (EC) cells or may exhibit altered pluripotency as well as karyotypically epigenetic changes. These alterations observed during enzymatic propagation of hBS cells are presumably due to an overly selective culture system in favor of cells specifically resistant to population pressure and stress, which usually starts to occur around passage 15-20.

Accordingly, development of a culture system allowing enzymatic dissociation of hBS cells into single cells even at high split ratios i.e. high proliferation rates when being passaged without resulting in the above-mentioned problems is desirable. Such a culture system is the subject of the present invention.

### BRIEF DESCRIPTION OF THE INVENTION

The present invention provides a culture system with a highly supportive culture environment for hBS cells which allows enzymatic dissociation down to single cells during each passage without compromising the cell line's undifferentiated, pluripotent and normal state over an extended period of time, such as, e.g., for more than 20 passages. The supporting environment of the herein presented culture system compensates for the selective stress inflicted at the cells during single cell passaging.

The culture system provided according to the present invention is a culture system for propagation of human blastocyst-derived stem (hBS) cells comprising

i) human feeder cells at a density of at least 50,000 cells/cm²,
ii) one or more dissociation agents for dissociation of hBS cell colonies into a single cell suspension, and
iii) a supportive culture medium,
which culture system makes it possible to propagate hBS cells by dissociation of hBS cell colonies into a single cell suspension at each consecutive passage for an extended time period, while maintaining the significant characteristics of hBS cells.

Furthermore, the present invention provides a method for propagation of hBS cells in a culture system according to the present invention, the method comprising the steps of

i) optionally, performing an adjustment procedure in order for the hBS cells obtained from a master cell line to adjust to the culture system,
ii) dissociating the hBS cells into a single cell suspension by the use of one or more dissociation agents,
iii) distributing the single cell suspension in a split ratio of at least 1:4, such as at least 1:5 into one or more culture vessels comprising human feeder cells at a density of at least 50,000 cells/cm², iv) incubating the hBS cells for from about 3 to about 25 days upon regular medium changes,
v) repeating n times from step ii), wherein n is an integer of at least 1, in order to propagate the hBS cells, while maintaining the significant characteristics of such cells.

In contrast to what has been disclosed in the prior art, the hBS cells only require a short adjustment procedure—if any at all—when transferred from a master cell line culture to the culture system of the present invention. Furthermore, the present invention solves the previously encountered problems relating to low stability and quality of the obtained hBS cells when propagated by enzymatic dissociation of hBS cell colonies into a single cell suspension at passage, as the hBS cells propagated in a culture system according to the present invention by a method according to the present invention can be propagated for more than 20 passages, such as, e.g., more than 30 passages, while maintaining the significant characteristics of hBS cells.

### DESCRIPTION OF THE INVENTION

As mentioned in the above, a main aspect of the present invention relates to a culture system allowing propagation of hBS cells by enzymatic dissociation of hBS cells into a single cell suspension without loosing the significant characteristics of hBS cells. The culture system for propagation of hBS cells according to the present invention comprises

i) human feeder cells at a density of at least 50,000 cells/cm²,
ii) one or more dissociation agents for dissociation of hBS cell colonies into a single cell suspension, and
iii) a supportive culture medium,
which culture system makes it possible to propagate hBS cells by dissociation of hBS cell colonies into a single cell suspension at each consecutive passage for an extended time period, while maintaining the significant characteristics of hBS cells. The hBS cells may be propagated in the culture system according to the present invention for more than 20 passages, such as, e.g., more than 25 passages, more than 30 passages, more than 35 passages, or more than 40 passages, and still maintain the significant characteristics of such cells.

One other aspect of the present invention is a culture system for propagation of hBS cells and subsequent separation, said system comprising:

i) human feeder cells at a density of at least 50,000 cells/cm²,
ii) one or more dissociation agents for dissociation of hBS cell colonies into a single cell suspension, and
iii) a supportive culture medium,
iv) magnetic particles for incorporation into the feeder cells,
which culture system makes it possible to separate the feeder cells from the hBS cells.

The separation efficiency of said system may be at least 50%, such as at least 70%, at least 80%, at least 90%, at least 99%, where separation efficiency is intended to mean the percentage of the total number of feeder cells that are attracted by the magnetic force applied.

As used herein, the term “propagation” is intended to mean that hBS cells are propagated in order to expand the hBS cell population, i.e. expand the amount of hBS cells. However, the culture system and the method for propagation described herein can also be used for the purpose of simply maintaining a hBS cell line.

As used herein, the term “significant characteristics of hBS cells” is intended to mean one, two, three, four, five, six, or seven of the following characteristics:

i) exhibits proliferation capacity in an undifferentiated state when grown on mitotically inactivated feeder cells, and/or
ii) exhibits stable chromosomal karyotype, i.e. no aberrations occurring during propagation of the hBS cells, and/or
iii) maintains potential to develop into derivatives of all types of germ layers both in vitro and in vivo, and/or
iv) exhibits at least two of the following molecular markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2, and/or
v) does not exhibit molecular marker SSEA-1 or other differentiation markers, and/or
vi) retains its pluripotency and forms teratomas in vivo when injected into immuno-compromised mice, and/or
vii) is capable of differentiating.

These characteristics may be analyzed as described in examples 7 and 8 at regular intervals such as every 1-20 passages, such as, e.g., every 5-15 passages or every 10 passages.

### Starting Material

The culture system of the present invention can be used for propagation of hBS cells or cell lines that are maintained in what is called a “master cell line” herein. As used herein a “master cell line” refers to the parent culture of a hBS cell line that is maintained in a traditional culture system using mechanical dissection of the hBS cell colonies at passage and a “master cell line” may also refer to a parent culture that has been vitrified.

### Feeders

A dense layer of feeder cells is required in a culture system according to the present invention in order to make said system sufficiently supportive to the hBS single cells, which are definitely more sensible towards the surrounding environment than are hBS cells in clusters. Accordingly, the density of human feeder cells in a culture system according to the present invention is from about 50,000 to about 500,000 cells/cm², such as, from about 50,000 to about 400,000 cells/cm², from about 50,000 to about 300,000 cells/cm², from about 50,000 to about 200,000 cells/cm², from about 60,000 to about 200,000 cells/cm², from about 70,000 to about 200,000 cells/cm². Normally, the feeder cells are seeded in the culture vessels from about 1 to about 10 days, such as, e.g., from about 1 to about 5 days, from about 2 to about 4 days, prior to seeding the hBS cells. Optionally, the culture medium may be changed at least one, such as, e.g., at least two, at least three, at least four or at least five times, prior to seeding the hBS cells.

Suitable human feeder cells for use in the culture system of the present invention may be derived from human tissue or they may be derived in vitro, such as, e.g., from hBS cells or cells derived from hBS cells.

The human tissue from which the human feeder cells may be derived include embryonic, fetal, neonatal, juvenile or adult tissue, and it further includes tissue derived from skin, including foreskin, umbilical chord, muscle, lung, epithelium, placenta, fallopian tube, glandula, stroma or breast. The human feeder cells may be derived from cell types pertaining to the group consisting of human fibroblasts, fibrocytes, myocytes, keratinocytes, endothelial cells and epithelial cells. Examples of specific cell types that may be used for deriving human feeder cells include embryonic fibroblasts, extraembryonic endoderm cells, extraembryonic mesoderm cells, fetal fibroblasts and/or fibrocytes, fetal muscle cells, fetal skin cells, fetal lung cells, fetal endothelial cells, fetal epithelial cells, umbilical chord mesenchymal cells, placental fibroblasts and/or fibrocytes, placental endothelial cells, post-natal human foreskin fibroblasts and/or fibrocytes, post-natal muscle cells, post-natal skin cells, post-natal endothelial cells, adult skin fibroblasts and/or fibrocytes, adult muscle cells, adult fallopian tube endothelial cells, adult glandular endometrial cells, adult stromal endometrial cells, adult breast cancer parenchymal cells, adult endothelial cells, adult epithelial cells or adult keratinocytes.

When human feeder cells are derived from hBS cells, the cells derived from hBS cells may be fibroblasts or have a mesenchymal phenotype.

In a specific embodiment of the present invention, the human feeder cells are fibroblasts, preferably derived from human neonatal foreskin fibroblasts. A human foreskin fibroblast feeder cell line can be established from skin samples from a circumcised baby boy by placing said skin samples in a culture vessel containing a suitable sterile culture medium, such as, e.g., a base medium, such as DMEM (Dulbecco's Modified Eagle's Medium) or IMDM (Iscove's Modified Dulbecco's Medium), supplemented with a mammalian serum, such as FBS or human serum, or supplemented with a serum replacement and 1% PEST (v/v). Preferably the culture medium is IMDM (Invitrogen) supplemented with 10% (v/v) human serum and 1% (v/v) PEST. A confluent monolayer of cells is obtained after from about 5 days to about 30 days. The feeder cells may now be passaged by enzymatic dissociation at intervals from about 2 days to about 10 days, such as, e.g., from about 4 days to about 9 days, from about 5 days to about 8 days, using one or more dissociating agents, such as, e.g. TrypLE™ Select. After establishment the feeders may be tested for a suitable selection of human pathogens in order to ensure their healthiness. Specifically, a cell line of human foreskin fibroblast feeder cells may be obtained as described in example 3 in the below.

In another embodiment the feeder cells used in a culture system according to the present invention may be commercially available feeder cells such as, e.g., hFF cells (American Type Culture Collection, CRL-2429 ATCC, Manassas, Va.) or human embryonic fibroblast cells (American Type Culture Collection, CCL-110 ATCC, Manassas, Va.).

Feeder cells used in the present invention may further be immortalized or genetically modified. Immortalization of feeder cells means the acquisition of the ability to grow through a theoretically indefinite number of divisions in culture. There are several methods for doing that and one approach is to transform the cells with e.g. viruses, retro viruses and/or by the expression of telomerase reverse transciptase protein (TERT). TERT is inactive in most cells, but when hTERT is exogenously expressed the cells are able to maintain telomere lengths sufficient to avoid replicative senescence.

Moreover, one of either cell type, i.e. the feeder cells or the hBS cells may be magnetically modified to allow separation of a mixed cell population thereof by applying a magnetic force. In a preferred embodiment of the present invention, the feeder cells used in the present invention are magnetically modified. Magnetic modification of the feeder cells may be performed by exposing the cell population to magnetic particles that can be incorporated into the cells by several means such as e.g. by electroporation, endocytosis or fagocytosis.

In addition the feeder cells may be genetically modified having specific genes integrated to the genome. These genes may code for markers of interest or for synthesis of biomolecules known to be beneficial to the hBS cells, such as growth factors, such as e.g. bFGF (basic fibroblast growth factor). These gene modifications can also induce apoptosis in the feeder cells and make induced removal of feeder cells available.

The human feeder cells used in the culture system of the invention have been growth inactivated in order to maintain a relatively fixed number of feeder cells to avoid that the feeder cells outgrow the hBS cells. Growth inactivation of human feeder cells may be performed by treating the cells with an anti-mitotic agent, such as, e.g., mitomycin, according to known methods or as performed in examples 1 and 4 in the below. Alternatively, the human feeder cells may be growth inactivated by subjecting the cells to a dose of irradiation, such as gamma irradiation sufficient to cause cell cycle arrest according to known methods.

Prior to growth activation of the feeder cells, they may be cultured in a culture medium supportive of the particular cell type of the feeder cells. When the feeder cells are human fibroblasts, the culture medium may be selected from the group consisting of Dulbecco's Modified Eagle's Medium (DMEM), Iscove's Modified Dulbecco's Medium (IMDM) supplemented with mammalian serum, such as human serum or FBS, or serum replacement and further supplemented with PEST. The feeders may be seeded directly in the culture vessel or seeded onto a matrix component in the culture vessel. The culture medium may be supplemented with serum, such as, e.g., FBS or human serum, in a concentration from about 1 to about 40% v/v, such as, e.g., from about 5 to to about 20% v/v or 10% v/v and/or with one or more antibiotics, such as penicillin and streptomycin. In a specific embodiment, culture medium is supplemented with penicillin-streptomycin in a concentration between from about 0.1% to about 10% v/v, from about 0.5% to about 2% v/v, or preferably 1% v/v. The feeder cells may be passaged in this culture medium at intervals ranging from about 2 to about 21 days, such as, e.g., from about 3 to about 10 days, from about 4 to about 8 days, such as every 7 day by use of one or more dissociation agents at split ratios between 1:2 to 1:20, such as, e.g., between 1:2 to 1:10, between 1:2 to 1:8. After approximately 2 to 10 passages, such as after 3 to 8 passages, the feeder cells may be growth inactivated as described above.

After growth inactivation the cells may be seeded in culture vessels, that are coated with a suitable matrix material, in a culture medium supportive of hBS cells, i.e. all media described in the below as supportive of hBS cell propagation are suitable. An example of a suitable matrix material may comprise recombinant human fibronectin, human placental extracellular matrix or gelatin, such as, e.g., recombinant human gelatin. A suitable concentration of gelatin is about 0.1% w/v. The feeder cells are seeded at a density of from about 50,000 to about 500,000 cell/cm², such as, e.g., from about 60,000 to about 200,000 cell/cm², or from about 70,000 to about 100,000 cell/cm². Ideally the feeder cells are used as feeder cells between passage 2 and 10, such as, e.g., between passage 3 and 8. Specifically, the feeder cells may be cultured as described in examples 1 and 3 in the below.

In a specific embodiment of the present invention, the feeder cells are xeno-free feeder cells, i.e. they have never been exposed to, directly or indirectly, material of non-human animal origin, such as cells, tissues, and/or body fluids and derivatives thereof. Example 3 and 4 in combination describes the establishment and cultivation of a xeno-free cell line of human foreskin fibroblast feeder cells.

### Dissociation Agents:

The present invention relates to a culture system, wherein the hBS cell colonies can be dissociated into a single cell suspension at passage. As used herein the term “single cell suspension” is intended to mean a suspension of hBS cells essentially comprising single cells, i.e., less than 10%, such as, e.g., less than 8%, less than 6%, less than 4%, less than 2%, or less than 1% of the cell entities may be clusters. Any remaining clusters may optionally be removed by use of a cell strainer, which functions as a mesh with a pore size whereby clusters are collected and single cells are let through. As mentioned in the above, this is a critical procedure since the single cell status is harsh on the hBS cells, why it has previously been found to imply complications with respect to cell survival and with respect to the stability and quality of the surviving cells.

When used in the context of a culture system according to the present invention, one or more dissociation agents may be an enzyme, a chelating agent or a combination of one or more enzymes and/or one or more chelating agents. Accordingly, the one or more dissociation agents may be a combination of at least two, such as, e.g. at least three, at least four, at least five, dissociation agents.

Enzymes that are suitable for use in the culture system of the present invention may include proteolytic enzymes and/or collagenolytic enzymes, such as, e.g., trypsin, trypsin-like, dispase, dispase-like, pronase, pronase-like, collagenase, collagense-like and matrix metalloproteinases. In particular suitable enzymes may be recombinant enzymes.

Suitable chelating agents may be chelators of divalent cations, such as, e.g., EDTA, EGTA and HEDTA.

Upon dissociation of the hBS cell colonies in step ii) a DNA'se may be added in addition to the one or more dissociation agents, which will prevent clumping caused by DNA released from disrupted cells.

Combinations of dissociation agents are particularly suitable for use in a culture system according to the present invention. Examples of such are TrypLE™ Select (Invitrogen), Accutase™ (Chemicon) and Accumax™ (Chemicon). TrypLE™ Select, which comprises a recombinant trypsin-like enzyme and EDTA, is free of animal components. Accutase™, which comprises proteolytic and collagenolytic activities and EDTA, contains no mammalian or bacterially derived components. In addition to the components of Accutase™, Accumax™ further comprises DNA'se activities.

Furthermore, the one or more dissociation agents or a combination thereof can be xeno-free, i.e. they have never been exposed to, directly or indirectly, material of non-human animal origin, such as cells, tissues, and/or body fluids and derivatives thereof.

### Culture Media

Culture media that are suitable for use in a culture system according to the present invention have to be highly supportive for the growth of hBS cells. A suitable culture medium may be a defined medium having a completely known composition. Suitable supportive culture media include supplemented base media, such as DMEM or IMDM. The supportive culture medium may be supplemented with one or more of the following constituents: mammalian serum, such as, e.g., FBS or human serum, KNOCKOUT® Serum replacement, penicillin, streptomycin, non-essential amino acids, L-glutamine, β-mercaptoethanol and hrbFGF (human recombinant basic fibroblast growth factor).

Also other media may be used in the present invention. Such medium may comprise salts, vitamins, an energy source (such as glucose), minerals, and amino acids. Suitable growth factors to be added to the medium could be e.g. GABA, pipecholic acid, lithium chloride, and transforming growth factor beta (TGFβ), and bFGF. Furthermore, such medium may be chemically defined.

The culture medium may also be a conditioned culture medium, i.e. a culture medium, which has been in contact with feeder cells, such as, e.g., human feeder cells, prior to being used as a culture medium for hBS cell propagation. A conditioned medium contains factors, which have been released from the feeder cells and as such it may be more supportive for hBS cell propagation than an unconditioned medium. Furthermore, the medium may be xeno-free, which is particularly relevant when the propagated hBS cells are to be used for any kind of medical application and consequently have to be of clinical standard.

The supportive culture medium may be supplemented with serum, such as, e.g., FBS or human serum, or alternatively a serum replacement, such as, e.g., KNOCKOUT® Serum replacement. The supportive culture medium may be supplemented with from about 1% to about 40% serum, such as, e.g., from about 5% to about 20% serum, such as with 10% serum.

The supportive culture medium may be supplemented with one or more growth factors selected from the group enabling to maintain the significant hBS cell characteristics, consisting of EGF (epidermal growth factor), HGF (hepatocyte growth factor), neurotrophins, fibroblast growth factors, such as acidic FGF and/or basic fibroblast growth factor (bFGF), preferably, human recombinant basic fibroblast growth factor (hrbFGF). In one embodiment, the supportive culture medium is supplemented with hrbFGF in a suitable concentration. A suitable concentration of hrbFGF may be within the range from about 0.5 to about 1000 ng/ml hrbFGF, such as, e.g., from about 1 to about 500 ng/ml hrbFGaF, from about 2 to about 200 ng/ml or from about 4 to about 1000 ng/ml hrbFGF.

In the following four suitable culture media are described. However, it is contemplated that other culture media can be used for as long as they provide the hBS cells with nutritional ingredients in a liquid form, i.e. inorganic ingredients such as trace elements and organic ingredients such as amino acids, salts, vitamins, energy providers, carbohydrates including sugars etc.

One suitable culture medium used in the invention is VitroHES™ medium supplemented with at least 4 ng/ml hrbFGF.

Another suitable medium that may be used in the invention is comprised of a DMEM or other base medium, such as KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine, 100 μM β-mercaptoethanol, at least 4 ng/ml hrbFGF.

Yet another suitable medium (hBS cell medium) that may be used in the invention is comprised as follows; KNOCKOUT® Dulbecco's Modified Eagle's Medium, supplemented with 20% KNOCKOUT® Serum replacement and the following constituents at their respective final concentrations: 50 units/ml penicillin, 50 μg/ml streptomycin, 0.1 mM non-essential amino acids, 2 mM L-glutamine and 100 μM, β-mercaptoethanol and further supplemented with at least 4 ng/ml hrbFGF.

Still another suitable culture medium used in the present invention is a xeno-free medium, which comprises a culture medium suitable for propagation of hBS cells. One suitable base medium is Dulbecco's Modified Eagle's Medium (DMEM). However other culture media may work as well. In addition to a xeno-free base medium, the xeno-free medium may further comprise human serum, hrbFGF, L-glutamine or glutamax, non-essential amino acids, β-mercaptoethanol, penicillin and/or streptomycin. The concentration of human serum in the xeno-free medium is preferably from about 1% v/v to about 30% v/v human serum, such as, e.g., from about 10% v/v to about 30% v/v human serum, from about 15% v/v to about 25% v/v human serum, and more preferably 20% v/v human serum. The concentration of hrbFGF in the xeno-free medium is preferably from about 2 ng/ml to about 100 ng/ml hrbFGF, such as, e.g., from about 5 ng/ml to about 50 ng/ml hrbFGF, from about 5 ng/ml to about 25 ng/ml hrbFGF, from about 5 ng/ml to about 15 ng/ml hrbFGF, such as, e.g., at least 4 ng/ml hrbFGF. The concentration of L-glutamine or Glutamax® in the xeno-free medium is preferably from about 0.5 mM to about 20 mM, such as, e.g., from about 0.75 mM to about 10 mM, from about 1 mM to about 5 mM, such as, e.g. 2 mM. The concentration of non-essential amino acids in the xeno-free medium is preferably from about 0.01 mM to about 1 mM, such as, e.g., from about 0.03 mM to about 0.8 mM, from about 0.05 mM to about 0.6 mM, from about 0.07 mM to about 0.4 mM, from about 0.09 mM to about 0.2 mM, such as, e.g. 0.1 mM. The concentration of beta-mercaptoethanol in the xeno-free medium is preferably from about 10 μM to about 200 μM, such as, e.g., from about 25 μM to about 175 μM, from about 50 μM to about 150 μM, from about 75 μM to about 125 μM, such as, e.g., 100 μM. The concentration of penicillin in the xeno-free medium is preferably from about 5 units/ml to about 200 units/ml, such as, e.g., from about 10 units/ml to about 150 units/ml, from about 25 units/ml to about 100 units/ml, from about 25 units/ml to about 75 units/ml, such as, e.g., 50 units/ml. The concentration of streptomycin in the xeno-free medium is preferably from about 5 μg/ml to about 200 μg/ml, such as, e.g., from about 10 μg/ml to about 150 μg/ml, from about 25 μg/ml to about 100 μg/ml, from about 25 μg/ml to about 75 μg/ml, such as, e.g., 50 μg/ml. In a specific embodiment, the culture medium is DMEM supplemented with 1-30% v/v human serum and 2-100 ng/ml hrbFGF. In one embodiment the base medium comprises 20% v/v human serum. In another embodiment the base medium comprises at least 4 ng/ml hrbFGF.

Superior quality human serum for use in the above-mentioned xeno-free culture medium has been repeatedly produced in our laboratory. The blood was tested for a number of standard pathogens at the Hospital's blood center (Hepatitis B, C, HIV, HTLV and syphilis). Accordingly, the human serum used in a xeno-free medium is preferably prepared by the following steps:
1) collecting healthy human blood in not-heparin coated bags,
2) agitating the not-heparin coated bags for a time period of from about 0.5 hours to about 5 hours, such as, e.g., from about 0.5 hours to about 2 hours,
3) incubating the not-heparin coated bags at a temperature of at the most 5° C. for a time period of at least 10 hours,
4) optionally, selection based on clotting quality such as, e.g., absence of non-clotted fibrin, opacity of the liquid phase,
5) separating the serum from the clotted material,
6) sterile filtrating the serum,
7) pooling serum from at least 15 donors,
8) freezing serum before use.

### Preferred Culture System

In a preferred embodiment the culture system of the present invention comprises

i) human neonatal foreskin feeder cells at a density of at least 50,000 cells/cm²,
ii) TrypLE™ Select for dissociation of hBS cell colonies into a single cell suspension, and
iii) VitroHES™ supplemented with at least 4 ng/ml hrbFGF as supportive culture medium,
which culture system makes it possible to propagate hBS cells by dissociation of hBS cell colonies into a single cell suspension at each consecutive passage for an extended time period, while maintaining the significant characteristics of hBS cells.

### Method for Propagation of hBS Cells

In another main aspect, the present invention relates to a method for propagation of hBS cells in a culture system as defined in the above, the method comprising the steps of

i) optionally, performing an adjustment procedure in order for the hBS cells obtained from master cell line to adjust to the culture system,
ii) dissociating the hBS cells into a single cell suspension by the use of one or more dissociation agents,
iii) distributing the single cell suspension in a split ratio of at least 1:4, such as at least 1:5 into one or more culture vessels comprising human feeder cells at a density of at least 50,000 cells/cm²,
iv) incubating the hBS cells for from about 3 to about 25 days upon regular medium changes,
v) repeating n times from step ii), wherein n is an integer of at least 1,
in order to propagate the hBS cells, while maintaining the significant characteristics of such cells.

One of the improvements provided by this method is that repeated passaging of hBS cells by enzymatic dissociation of the hBS cell colonies into a single cell suspension, while maintaining the significant characteristics of hBS cells, is rendered possible. Accordingly, n may be at least 5 such as, e.g., at least 10, at least 15, at least 20, at least 25, at least 30, at least 35, or at least 40. In order to verify, that the significant characteristics of the hBS cells are maintained before repeating from step ii), i.e. before passaging the hBS cells, the method may further comprise the steps of vi) analyzing the cells obtained in step iv) to see whether the significant characteristics of hBS cells are maintained,

vii) repeating from step ii) if the significant characteristics of hBS cells are maintained.

### Dissociation Procedure

The dissociation of hBS cells into a single cell suspension in step ii) may be performed by treating the hBS cell colonies with one or more dissociation agents. After removal of the used medium, the hBS cell colonies may be washed in PBS, which may be depleted for calcium and magnesium. One or more dissociation agents are added to the colonies and allowed to work for a suitable time, i.e. until the outer regions of the hBS cell colonies start to round up from the feeder layer, by a suitable temperature, such as, e.g., 37° C. When the one or more dissociation agents comprises one or more enzymes, a suitable amount of enzyme activity ranges from about 10 Units/mL to about 5000 Units/mL, such as, e.g., from about 100 Units/mL to about 500 Units/mL. After the incubation, repeated trituration with a pipette may be performed in order to assist the one or more dissociation agents in breaking apart the cell sheet in order to obtain hBS single cells. As the presence of the one or more dissociation agents may negatively affect the capability of the hBS cells to form colonies once seeded onto the human feeder cells in step iii), the effect of the one or more dissociation agents may be diminished prior to step iii). The effect of the one or more dissociation agents can for example be diminished by physical removal, for example by centrifugation, filtration or sedimentation in order to separate the hBS cells from the one or more dissociation agents, dilution of the one or more dissociation agents, addition of one or more inhibitors of the one or more dissociation agents or addition of one or more substrates of the one or more dissociation agents in excess. Alternatively, the one or more dissociation agents may be capable of auto-inhibition, i.e. they have an inherent capability of inhibiting their own function after a certain time period, as is for example the case for Accutase™ and Accumax™. After removal of the one or more dissociation agents, the obtained hBS single cells are resuspended in a supportive culture medium according to the invention, in order to obtain a single cell suspension of hBS cells.

### Split Ratio

In step iii) of the method described above, the hBS cells in the obtained single cell suspension may be distributed into one or more culture vessels comprising human feeder cells prepared as described in the above, i.e. the hBS cells are seeded on the feeder cells. The important feature of this seeding is, that the present method and culture system allow cells to be distributed in a split ratio of at least 1:4, such as at least 1:5 which means that the hBS cells are distributed onto an area of feeder cells that is 5 times larger than the area the hBS cells were dissociated from prior to step ii). The criteria for choosing a certain split ratio depend upon confluency, growth rate and homogeneity of the hBS cell colonies in the culture vessel based on morphological inspection and cell counts after dissociation. The higher the above factors, the higher split ratio can be employed without compromising the stability and quality of the cells, and the higher expansion of the number of hBS cells is achieved. Suitable split ratios may be within a range from about 1:4 or 1:5 to about 1:5000, such as, e.g., from about 1:20 to about 1:1000, from about 1:50 to about 1:500, where an often used split ratio is about 1:20.

### Incubation

After seeding the hBS cells at a certain split ratio, the cells are incubated at about 37° C. in a humid atmosphere, i.e. preferably about 95% humidity, for from about 3 to about 25 days, such as, e.g., from about 4 to about 20 days, or from about 6 to about 12 days. During the incubation in step iv), the culture medium may be changed at regular intervals from about 1 to about 14 times a week, such as, e.g., from about 2 to about 6 times a week, from about 2 to about 4 times a week, such as 3 times a week.

### Adjustment Procedure

Since the enzymatic dissociation of hBS cell colonies into a single cell suspension, which is an important element of the present invention is a major change for the hBS cells associated with stress, potential low adhesion and subsequent low proliferation, spontaneous differentiation, and even potential cell death, it may be necessary to let the hBS cells adjust to the culture system of the present invention by subjecting them to milder conditions for one or more passages. By milder conditions is meant a smaller split ratio and a longer incubation time than prescribed in the method for propagation itself. Accordingly, the adjustment procedure according to the present invention comprises the steps of

a) dissociating the hBS cell colonies into a single cell suspension by use of one or more dissociation agents,
b) distributing the single cell suspension in a split ratio of at least 1:3 into one or more culture vessels comprising human feeder cells at a density of at least 50,000 cells/cm²,
c) incubating the hBS cells for from about 5 to about 30 days, such as, e.g., from about 7 to about 16 days or from about 7 to about 10 days, upon medium changes at regular time intervals,
d) optionally, repeating from step a) at the most 5 times,
in order to obtain homogeneous undifferentiated hBS cells colonies. By homogeneous colonies is intended to mean colonies having an even cell density throughout the colony area without piling-up structures.

The dissociation in step a) and the medium changes in step c) are performed essentially as the dissociation in step ii) and step iv) of the method for propagation described in the above.

One of the improvements provided by the present invention is however, that the necessary adjustment procedure—if needed at all—is short and can be performed on many today existing hBS cell lines established and cultured according to e.g. WO03055992. Accordingly, if step d) is included it may be performed at the most 5 times, such as, e.g., at the most 4 times, at the most 3 times, at the most 2 times.

In a preferred embodiment of the present invention, the method for propagation of hBS cells in a culture system as defined in the above comprises the steps of

i) performing an adjustment procedure comprising the steps of a) dissociating the hBS cell colonies into a single cell suspension by use of one or more dissociation agents, b) distributing the single cell suspension in a split ratio of at least 1:3 into one or more culture vessels comprising human feeder cells at a density of at least 50,000 cells/cm², c) incubating the hBS cells for from about 5 to about 30 days, such as, e.g., from about 7 to about 16 days or from about 7 to about 10 days, upon medium changes at regular time intervals, d) repeating from step a) at the most 5 times, in order to obtain homogeneous undifferentiated hBS cells colonies,
ii) dissociating the hBS cells into a single cell suspension by the use one or more dissociation agents, such as, e.g., TrypLE™ Select,

iii) distributing the single cell suspension in a split ratio of at least 1:4, such as at least 1:5 into one or more culture vessels comprising human feeder cells at a density of at least 50,000 cells/cm²,

iv) incubating the hBS cells for from about 3 to about 25 days upon regular medium changes,
v) repeating at least 20 times from step ii),
in order to propagate the hBS cells, while maintaining the significant characteristics of such cells.

### Separation

A suitable method for separation of feeder cells and hBS cells from each other according to the present invention may comprise the following steps:

i) exposing feeder cells to magnetic particles to obtain magnetically modified feeder cells prior to seeding;
ii) after an optional medium change, seeding and subsequent culturing the hBS cells on the magnetically modified feeder cells for at least 1 day;
iii) detaching the mixed cell population of feeder cells and hBS cells from the culture vessel and dissociating said population into a single cell suspension by the use one or more dissociation agents, such as, e.g., TrypLE™ Select, and
iv) separating the feeder cells from the hBS cells by applying a magnetic force.

The separation efficiency in said method may be at least 50%, such as at least 70%, at lest 80%, at least 90%, at least 99%, where separation efficiency is intended to mean the percentage of the total number of feeder cells that are attracted by the magnetic force applied.

### Analysis of Significant Characteristics

In order to investigate, whether the hBS cells obtained according to the present invention maintain the significant characteristics of hBS cells, Cellartis' hBSC lines SA001 and SA121 were transferred to a culture system according to the present invention and cultured according to the method of the present invention for 20-40 consecutive passages. At passage 20 and above the hBS cells were characterized thoroughly. Morphology of individual cells and colony morphology was evaluated microscopically revealing normal morphology for hBS cells and colonies thereof (FIG. 1). Expression of markers was analyzed on the protein expression level using immunohistochemistry/histochemistry (Oct-4, SSEA-3, SSEA-4, Tra1-60, Tra1-81, SSEA-1) (example 7, FIG. 2 and FIG. 5). Genetic characterization as performed by karyotyping and FISH analysis revealed maintained karyotype for more than 20 passages (example 8, FIG. 3 and FIG. 5). Pluripotency was evaluated for hBS cells obtained according to the present invention by the formation of teratomas in SCID mice, revealing the hBS cells to be pluripotent undifferentiated hBS cells after more than 20 passages (example 9, FIG. 4 and FIG. 5).

Additional examples of characterization of hBS cells cultured according to the present invention may be analysis of clonal survival, such as performing colony formation assays of equivalent numbers of single cells seeded in different culture combinations or parameters, such as e.g. feeder types and densities, dissociation agents, and exposure times to dissociation agent. Two important parameters identified in the present invention are i) the choice of dissociation agent, which tend to be of importance for number of colonies formed and ii) the choice of feeder type, which tends to be of importance for the quality of the colonies formed in terms of being undifferentiated. Grade of differentiation may in turn be analyzed by morphology in a microscope and potentially correlated to previously performed analysis of marker expressions for known undifferentiated and differentiated markers.

Stem cells, and blastocyst-derived stem cells may further be characterized for their activity of the enzyme telomerase, which can be tested for with e.g. a kit called Telomerase PCR ELISA kit (Roche). The kit uses the internal activity of telomerase by amplification of the product by polymerase chain reaction (PCR) and detection of it with an enzyme linked immunosorbent assay (ELISA). Telomerase activity may as well be measured by QPCR.

The differentiation status of the cells in the present invention can furthermore be tested by QPCR for specific genes. In the following is shortly described how this can be done: Undifferentiated or differentiated hBS cell colonies may be detached from the culture plate mechanically as whole colonies and washed in PBS and stored in −80° C. RNA may further be extracted using e.g. Qiagen RNeasy Mini Kit according to the manufacturer's instructions. Reverse transcription is performed using a suitable kit, such as Bio-Rad iScript First Strand Synthesis Kit (according to the manufacturer's instructions) in a Rotorgene 3000 (Corbett Research) and the QPCR is performed under suitable conditions. All genes may be quantified in the same run and—if possible—differentiation status of several samples can be compared by calculating mathematical indices for the individual samples based on the genetic markers. (More detailed protocols are described in WO2006094798.)

The individual components used in the herein presented invention, such as the feeder cells, the medium, and the blastocyst may prior to use, as well as the hBS cell lines cultured according to the present invention, be tested for human pathogens, such as e.g. Mycoplasma, Human Immunodeficiency Virus type 1 and 2, Hepatitis B and C, Cytomegalovirus, Herpes Simplex Virus type 1 and 2, Epstein-Barr Virus, and Human Papilloma Virus. The absence of human pathogens is of importance for any clinical use of hBS cell line and differentiated cells or other biological material derived from such cell lines.

### Further Aspects of the Invention

In the further aspects of the present invention described in the below, the details and particulars discussed under the main aspects above shall apply mutatis mutandis.

### Further Procedures

The hBS cells obtained according to the present invention may be subjected to further procedures for manipulation and/or analysis of hBS cells. For example, a hBS cell line obtained according to the present invention can be used for the preparation of differentiated cells. Furthermore, a hBS cell or cell line according to the invention is capable of undergoing freezing and thawing. In a specific embodiment, the hBS cell line obtained in the present invention can be frozen and thawed according to a vitrification method previously presented by Cellartis, WO2004098285. To increase homogeneity of the hBS cell cultures, the cells obtained in the present invention can be subject for clonal derivation as described in WO2005059116. Also, the hBS cells obtained according to the present invention, may be transferred to a feeder free culture system as described in WO2004099394. A QPCR method for determination of the state of differentiation of e.g. hBS cells described elsewhere by the applicant may be employed on hBS cells obtained according to the present invention, or derivatives thereof, such as, e.g. cells that have been obtained according to the present invention and subsequently subjected to cell differentiation procedures. The patent applications referred to in this paragraph are hereby incorporated by reference.

Dissociation of hBS cell colonies into single cells at passage, made possible by the present invention, provides several improvements over existing culture systems and methods. In the following several applications for which hBS cells obtained according to the present invention is particularly suitable are outlined to further emphasize the benefits and improvements provided by the present invention.

The present invention facilitates propagation of hBS cells compared to existing culture systems and methods due to the large split ratios made possible by the present invention, allowing a higher degree of expansion of the amount of hBS cells in comparison with existing methods. This would imply, that the present invention provides improved possibilities of up-scaling the expansion of hBS cells. The combination of obtaining single cell suspensions at passage and the high split ratios made possible according to this invention enables scalable production of hBS cells, which may furthermore be subject to automatization. Accordingly, scalable production may be achieved by complete or partial automatization of the procedure according to the invention, thereby providing a time- and cost-saving culture system and method for propagation of hBS cells.

Accordingly, one embodiment of the present invention relates to scaleable production of hBS cells using the culture system for and/or the method for propagation of hBS cells disclosed herein. The one or more dissociation agents used in this embodiment of the invention may include at least one of TrypLE™M Select, Accutase™ and Accumax™. Another embodiment of the present invention is the use of TrypLE™ Select, Accutase™ and/or Accumax™ for scalable production of hBS cells. The production and manipulation of hBS cells for scaled up culture may use novel culture systems for bulk culture, such as multiwell plates, multilayer flasks and bioreactor modules. The preparation, treatment and analysis of hBS cells or cells derived from hBS cells in multi-well format plates, multilayer flasks or bioreactor modules, according to our system does not require manual selection or micromanipulation and can therefore be scaled up and automated using robotization. Suitable robots could be based on XYZ dispensing heads which allow pipetting to and from culture vessels such as liquid handling stations or could be based on a robotic arm which mimics the movements of a human being during culture vessel and pipette handling. Within the robotic system environmental parameters such as e.g. temperature, nutrient supply, pH, pressure, shear forces and oxygen should be maintained within optimal limits.

The attainment of hBS cells as single cells in the single cell suspension obtained according to the present invention, enables exact quantification of hBS cells using known cell quantification procedures and devices, such as, e.g., the NucleoCounter, Hemocytometer Manual Count, Flowcytometer Automated Count. Such quantification is important in order to be able standardize and improve all types of procedures the hBS cells may be subjected to.

Furthermore, having the hBS cells as single cells at passage further enable these cells to be subjected to different cell separation or cell sorting techniques known in the art, such as, e.g., density gradient media, antibody based chromatography or antibody coated magnetic beads, for example in order to separate hBS cells from remnants of feeder cells. Alternatively, the single cell suspension of hBS cells obtained in step ii) may be subjected to different kinds of sorting techniques, such as, e.g., FACS (fluorescent automated cell sorting) or magnetic bead sorting, density gradient centrifugation, (affinity) chromatography separation, for example in order to separate transfected hBS cells from un-transfected hBS cells.

hBS cells in single cell solution as described and generated in the present invention may be a good starting point for limiting dilution cloning to generate clones with unique features from hBS cell lines.

Moreover, to further facilitate use of hBS cells cultured according to the present invention the hBS cells may be separated from the feeder cells. In the following, one such separation method is described, without the intention of limiting the scope to other potential methods.

One potential approach for separation of hBS cells from feeder cells may be to allow incorporation of small iron particles into one of the cell types. Such incorporation may be performed spontaneously by the cells, such as by e.g. endocytosis or fagocytosis or by electroporation. The cells may be exposed to suitable iron particles in suspension in the culture medium prior to seeding. Feeder cells may prior to exposure to the iron particles be mitomycin C treated or in an alternative way mitotically arrested. The iron particles may be of several types or brands. They may further be of Fe2+ ions or Fe3+ ions or a mix thereof. In one embodiment of the present invention Endorem™ is used. The iron particles may further have a dimension of 1.0 nm to 50 nm in diameter, such as e.g. between 2.0 and 40 nm, between 4.0 and 30 nm. The concentration of the iron particle may range from between 0.1 ug/ml and 560 ug/ml, such as from between 0.2 and 300 ug/ml, from 0.5 and 100 ug/ml, from 0.75 to 10 ug/ml, from 1.0 to 3.0 ug/ml. The cells may be exposed to the iron containing solution for from around 1 minute to about 48 hours, such as from about 20 minutes to about 12 hours, such as from about 60 minutes to about 5 hours, such as from about 2 to 3 hours. After exposure to the magnetic particles the feeder cells may be seeded in a culture vessel and the medium exchanged. A single cell solution of hBS cells may be seeded on the feeder cells as soon as a layer has been formed and up to at least one week after seeding of the feeder cells. The hBS cells may be kept in culture on the iron containing feeder cells for at least 1 day, such as at least 2, at least 4, at least, 7, at least 10, at least 20 days.

The separation may further be performed by producing a single cell suspension of the mixed cell populations as described above and subsequently exposing said cell suspension to a magnetic force. The magnetic force may originate from any suitable magnet compatible in dimension with the vessel or tube in which the cells subject to separation are being kept. One potential outline of the separation is described in Example 13.

The hBS cells obtained according to the present invention are also particularly suitable for being subjected to cell transfection procedures, since the single cell status of the hBS cells avoids cell fusion during electroporation, thereby avoiding mixed clones and improving the efficiency of transfection. Accordingly, in one embodiment of the present invention the obtained hBS cells are subjected to cell transfection procedures, such as, e.g., by use of viral agents, lipofectamin, electroporation, calcium phosphate mediation, in order to obtain genetically modified hBS cells. Genetic modification of hBS cells is useful for several applications such as, e.g. for use as reporter genes, for knock-in and knock-outs to be used in developmental assays and tests in e.g. drug discovery and for toxicity testings as well as for use as disease models.

Furthermore, the hBS cells obtained according to the present invention are suitable for use in multiwell plate assays, since the obtained single cell suspension is easily distributed homogeneously in a multiwell plate. Accordingly, the single cell suspension of hBS cells present invention may be used in a multiwell plate assay for example in order to perform toxicity testing of different chemical compounds or in a drug discovery procedure for identification of drug candidates.

### A Single Cell Suspension

The present invention further relates to a single cell suspension of hBS cells, which single cell suspension is capable of surviving and maintaining the significant characteristics of hBS cells for more than 20, such as, e.g., more than 25, more than 30, more than 35 or more than 40 passages, when subjected to a method for propagation of such cells as described in the above.

### A hBS Cell Line

The present invention further relates to a hBS cell line, which hBS cell line is capable of surviving and maintaining the significant characteristics of hBS cells for more than 20, such as, e.g., more than 25, more than 30, more than 35 or more than 40 passages, when subjected to a method for propagation of such cells as described in the above.

### Seeding of Single Cell Dissociated hBS Cells on Low Feeder Density for Particular Usage

In some cases it may be advantageous to seed the cells in the single cell suspension of the present invention onto feeder cells, wherein the feeder cells are present at a low density such as below about 50,000 cells/cm2, about 10,000 cells/cm2, about 15,000 cells/cm2, about 20,000 cells/cm2, about 25,000 cells/cm2. This may be relevant e.g. as an intermediate step, i.e. the last or final step of the procedure described in the invention, before use in differentiation applications for toxicity testing or in systems for derivation differentiated cells types from hBS cell lines. In such applications, a low feeder density may be advantageous when hBS cells are separated from the feeders cells.

### Kit According to the Present Invention

In one embodiment, the invention relates one or more kits comprising one or more of the components of a culture system according to the present invention. Accordingly, a kit according to the present invention comprises at least one, such as, e.g., at least two of the following components

i) a single hBS cell population
ii) a user manual describing a method for propagation of the hBS cells.

Furthermore, the invention also relates to a kit comprising a first component comprising

i) a single cell population and at least one, such as, e.g., at least two or at least three of the following components
ii) human feeder cells,
iii) one or more dissociation agents for dissociation of hBS cell colonies into a single cell suspension, and
iv) a supportive culture medium.

In the above kits the single cell population may be derived from a xeno-free derived hBS cell line and one or more of any other components ii)-iv) may be xeno-free.

Preferably, further components of a kit according to the present invention may be a user manual describing a method for separation in accordance with the details and particulars described for the method of the present invention and/or a magnet.

### FIGURE LEGENDS

FIG. 1:

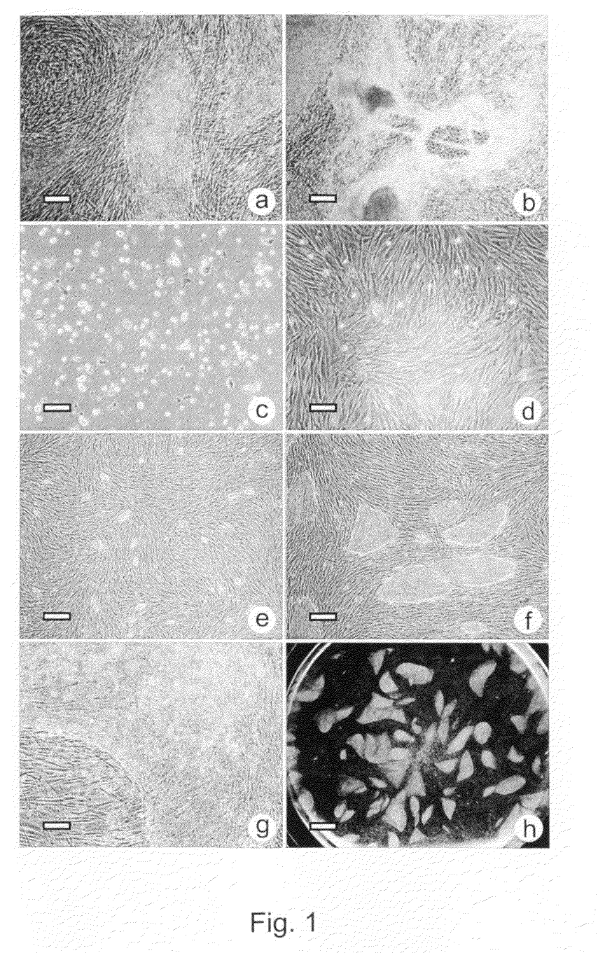

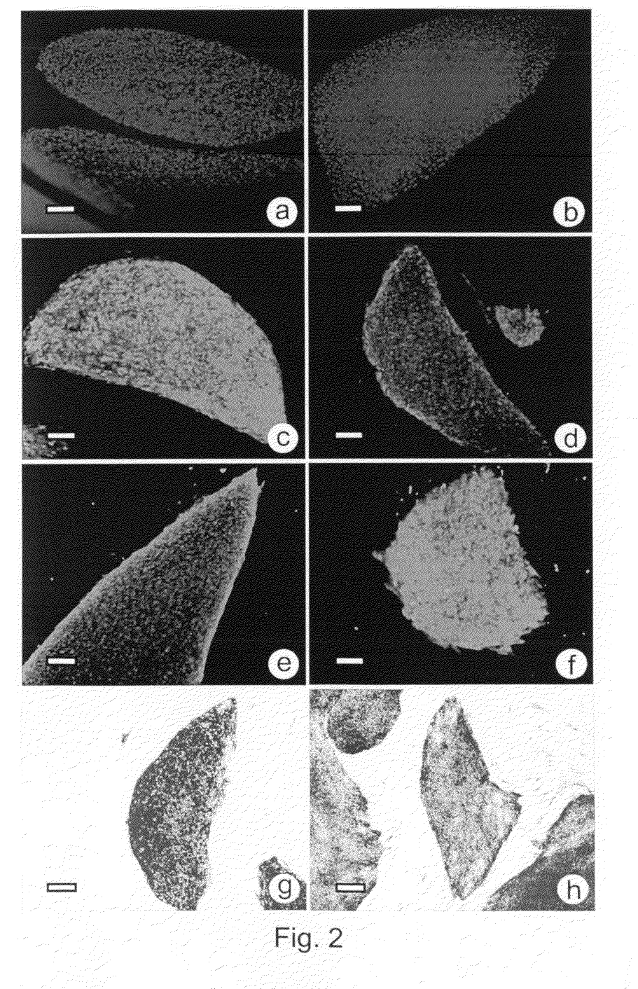

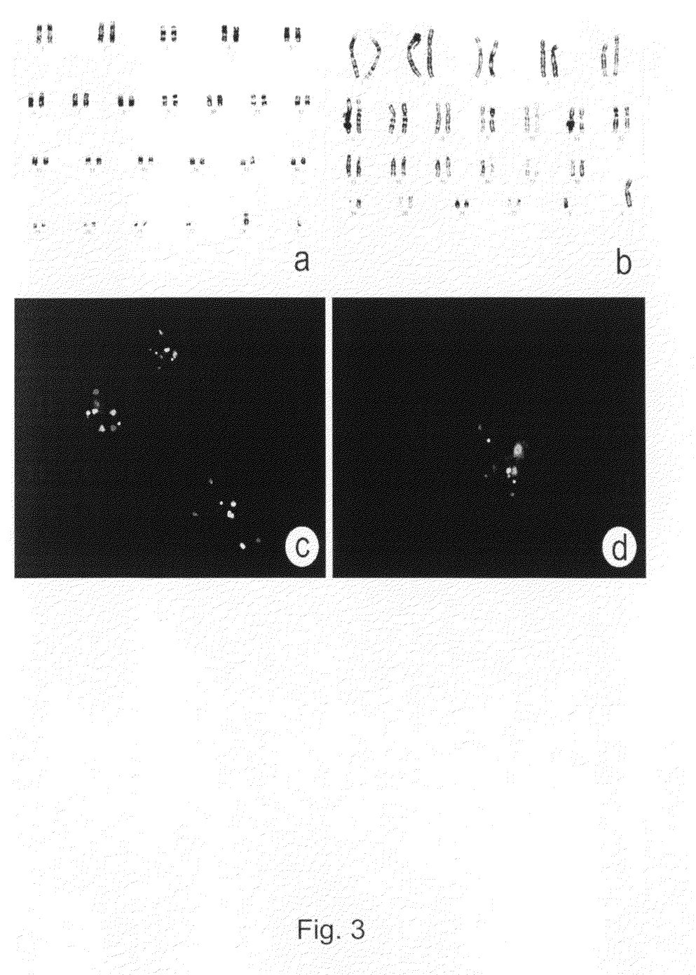

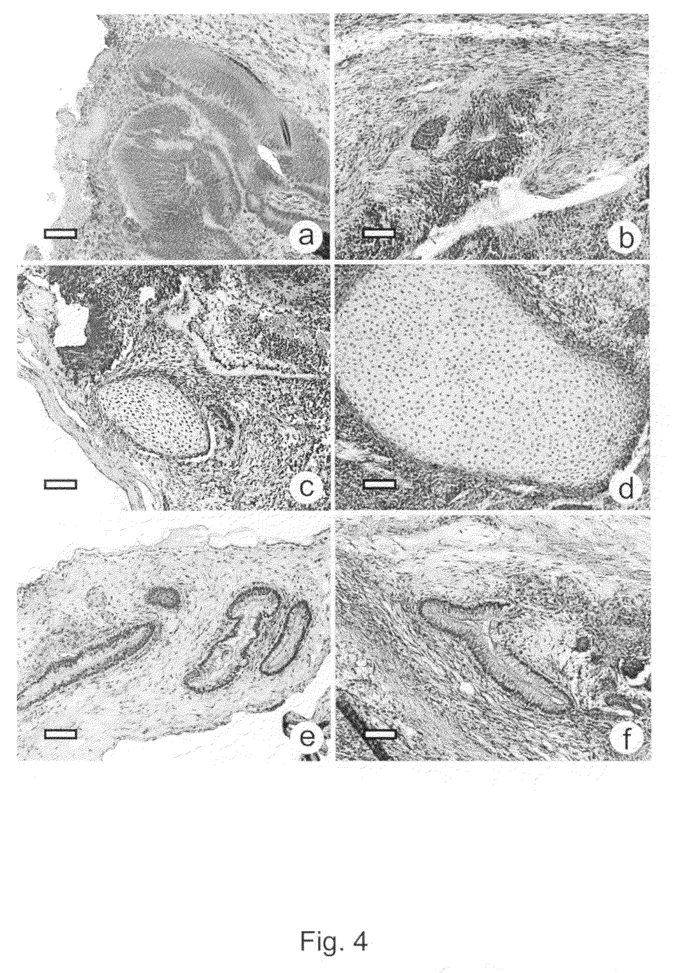

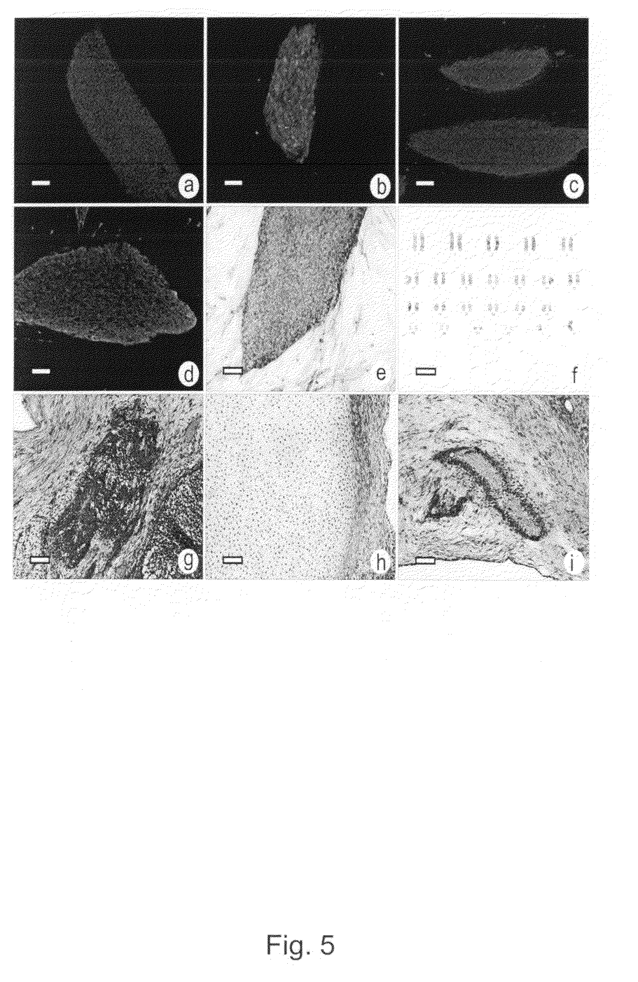

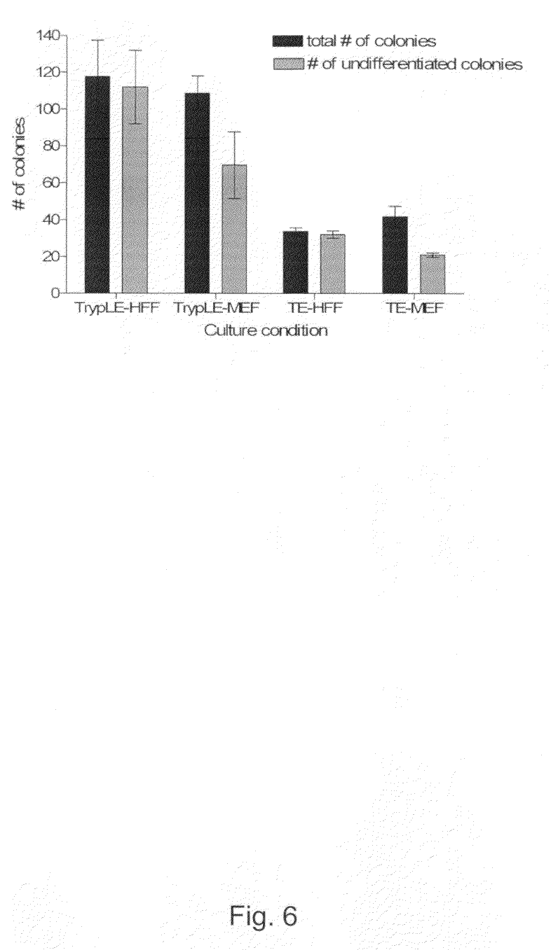

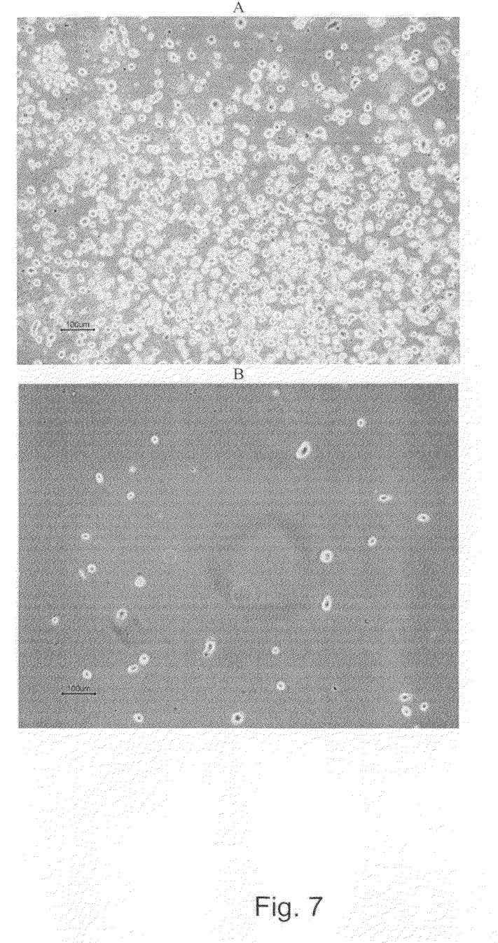

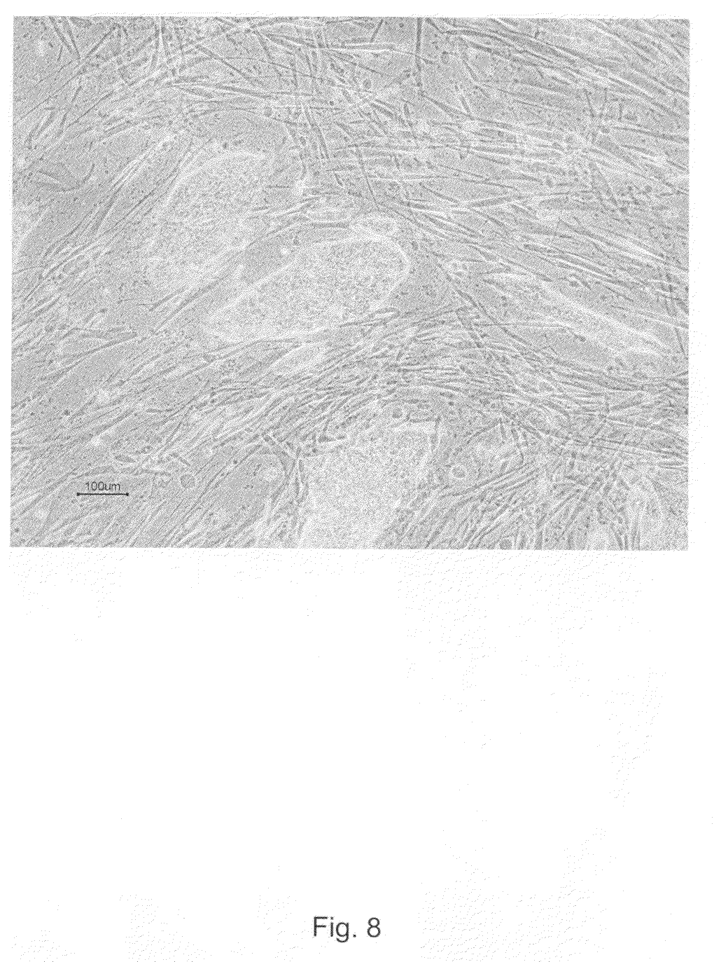

Morphology of hBS cell colonies cultured and passaged as single cells using hFFs and enzyme (TrypLE Select™).

a) Adapting hBS cells colonies after their first enzymatic dissociation.
b) Areas with heterogeneous cell population observed during early adjustment (the first passages in the new system).
c) Cluster size, i.e. single cells during enzymatic passage.
d) Single cell suspension of hBS cells on hFFs.
e) hBS cells adjusted to the present system, two days after enzymatic dissociation.
f) hBS cell colonies adjusted.
g) Homogenous hBS cell colony (to the upper right).
h) Culture well with hBS cell colonies, overview.
Scale bar 50 μm (c), 100 μm (a,b,g), 200 μm (d), 250 μm (e,f, 1.66 mm (h).

FIG. 2:

In the following figure legends “SA001 TrypLe” refers to hBS cells from Cellartis's cell line SA001, which have been propagated using TrypLE Select™ and “SA002 TE” refers to hBS cells from the same cell line, which have instead been propagated using Trypsin EDTA. Immunohistochemical staining of SA001 after more than 20 enzymatic passages using TrypLE Selectm and Trypsin EDTA:

SA001 TrypLe stained for Oct-4 (a), TRA-1-81 (c), SSEA-4 (e) and alkaline phosphatase (g). SA001 TE stained for Oct-4 (b), TRA-1-81 (d), SSEA-4 (f) and alkaline phosphatase (h).
Scale bar 100 μm (a-g). Scale bar 250 μm (g).

FIG. 3:

Karyotypes & FISH of SA001 after 25 passages using TrypLE Select™

(a) The chromosomes from SA001 TrypLE were diploid normal. The figure shows a representative karyotype. (b) SA001 TE, diploid normal (c,d) FISH analysis of selected chromosomes from SA001 TrypLE (c) and SA001 TE (d) demonstrated that the cells were XY and diploid normal for chromosomes X (blue), Y (gold), 13 (red), 18 (aqua) and 21 (green).

FIG. 4:

In vivo Pluripotency test of SA001. Teratomas after more than 20 passages using TrypLE Select™: and Trypsin-EDTA.

Histological analysis of teratomas from SA001 TrypLE (a,c,e) and SA001 TE (b,d,f) after 27 respectively 22 enzymatic passages. (a, b) Neuroectoderm (ectoderm), (c,d) Cartilage (mesoderm), (e,f) Secretory epithelium (endoderm)

Scale bars 25 μm (a,c,e) and 50 μm (b,d,f).

FIG. 5:

Characterization of SA121 after 20 enzymatic single-cell passages using TrypLE Select™.

(a) Oct-4 immunohistochemical staining, (b) TRA-1-60, (c) SSEA-3, (d) SSEA-4, (e) alkaline phosphatise. (f) Diploid normal karyotype of SA121 TrypLE after 20 single-cell enzymatic passages. (g-i): Teratomas derived from SA121 after 23 passages (g). Neuroectoderm (ectoderm) (h). Cartilage (mesoderm). (i). Secretory epithelium (endoderm). Scale bars: a-e: 100 μm, g-i: 50 μm.

FIG. 6:

Single cell enzymatic passaging with TrypLE™ Select results in increased clonal survival. When cells were transferred to hFFs after TrypLE™ Select treatment it resulted in a 3-fold increase in the number of hBS cell colonies formed compared to Trypsin-EDTA treatment (p=00.1). If dissociated hBS cells were plated on hFFs, significantly increased numbers of good colonies were obtained (p=0.2) compared to if they were plated on mEFs. The data are presented as the mean plus standard error (n=3).

FIG. 7

hFF cells after one week in culture. (A) shows the hFFs collected on the magnet and (B) shows the few remaining hFF cells in suspension.

FIG. 8

Single cell enzymatic passaging with TrypLE™ Select performed on human embryonic fibroblast cells (American Type Culture Collection, CCL-110 ATCC, Manassas, Va.)

### EXAMPLES

### Example 1

### Culture of Human Foreskin Fibroblast Feeders and Use as a Feeder Layer

Commercially available hFFs were obtained from the American Type Culture Collection (CRL-2429 ATCC, Manassas, Va.) and were cultured in Iscove's DMEM (Gibco Invitrogen Corporation, Paisley, Scotland; http://www.invitrogen.com), supplemented with 10% of FBS (Invitrogen) and 1% penicillin-streptomycin. The cells were passaged regularly (weekly) at a split between 1:2 and 1:8 using Trypsin-EDTA (Invitrogen). Confluent monolayer of hFF were treated with mitomycin-C (Sigma) (10 μg/ml for 2.5 hrs) and plated on 0.1% gelatin (Sigma) coated IVF dishes at a density of 70,000-80,000 cells/cm² in VitroHES™ medium supplemented with 4 ng/ml human recombinant basic fibroblast growth factor (hrbFGF, Invitrogen). hFF cells were used as feeders between passage 4 and passage 12.

### Example 2

### Culture of Human Embryonic Fibroblast Cells and Use as a Feeder Layer

Commercially available human embryonic fibroblast cells were obtained from the American Type Culture Collection (CCL-110 ATCC, Manassas, Va.) and were cultured in Iscove's DMEM (Gibco Invitrogen Corporation, Paisley, Scotland; http://www.invitrogen.com), supplemented with 10% of FBS (Invitrogen) and 1% penicillin-streptomycin. The cells were passaged regularly (weekly) at a split between 1:2 and 1:8 using Trypsin-EDTA (Invitrogen). Confluent monolayer of human embryonic fibroblast cells were treated with mitomycin-C (Sigma) (10 μg/ml for 2.5 hrs) and plated on 0.1% gelatin (Sigma) coated IVF dishes at a density of 70,000-80,000 cells/cm² in VitroHES™ medium supplemented with 4 ng/ml human recombinant basic fibroblast growth factor (hrbFGF, Invitrogen). Human embryonic fibroblast cells were used as feeders between passage 4 and passage 12, FIG. 8.

### Example 3

### Establishment of a Human Foreskin Fibroblast Feeder Cell Line (Such as Cell Line hFF003)

Human foreskin samples were aseptically collected in sterile IMDM (Invitrogen) containing 2× Gentamycin from a circumcised 8 week old boy. Skin explants were placed inside 25 cm² primaria tissue culture flasks (Becton Dickinson) containing IMDM medium (Invitrogen), 1% penicillin-streptomyocin (Gibco Invitrogen Corporation) and 10% of human serum. After approximately 10 days, a confluent monolayer was established. The cells were serially passaged using TrypLE™ Select (Invitrogen). After expansion they were tested for a standard panel of human pathogens (mycoplasma, HIV of type 1 and 2, Hepatitis of type B and C, Cytomegalovirus, Herpes Simplex Virus type 1 and 2, Epstein-Barr virus, Human Pailloma virus) all resulting negative.

### Example 4

### Feeder Layer Preparation from the In-House Established Foreskin Fibroblast Cell Line

Prior to plating the xeno-free human fibroblast feeders, the tissue culture treated wells were coated with 0.1% recombinant human gelatin (Fibrogen) for a minimum of 1 hour at room temperature. Confluent monolayers of xeno-free hFF003 (fifth to eight passage) cells grown in IMDM, 10% human serum and 1% penicillin-streptomyocin were then treated with mitomycin-C (Sigma) for (10 μg/ml, 2.5 hours). Mitomycin-C treated feeders were plated on IVF wells (Becton Dickinson), 200,000 cells per 2.89 cm² in a medium which was based on DMEM (as above) supplemented with 10% (v/v) human serum, 1% penicillin-streptomyocin, 1% Glutamax, 0.5 mmol/l β-mercaptoethanol and 1% non-essential amino acids (Gibco Invitrogen Corporation). Prior to the placing blastocysts with their inner cell mass cells and cells derived therefrom or hBS cells, the medium was changed to a DMEM (as above), now instead supplemented with 20% (v/v) human serum, 4 ng/mL hrbFGF, 1% penicillin-streptomyocin, 1% Glutamax, 0.5 mmol/l β-mercaptoethanol and 1% non-essential amino acids (Gibco Invitrogen Corporation). (Same medium as described in Example 3.)

### Example 5

### Transfer of hBSC to Enzymatic Propagation Culture

The hBS cell lines SA001, SA002, SA002.5, SA121, SA167, SA348, SA461 and SA502 (Cellartis AB, Göteborg, Sweden, http://www.cellartis.com) had been established and characterized as previously described [Heins, Noakssson] and in WO03055992. Such material can be obtained from Cellartis AB and is also available through the NIH stem cell registry http://stemcells.nih.gov/research/reistry/. Cellartis AB has two hBS cell lines (SA001 and SA002) and one subclone of SA002 (SA002.5) available through the NIH. Those hBS cell lines have been frequently used in the present invention. All the hBS cell lines used are approved and registered by the UK Stem Cell Bank Steering Committee and SA001, SA002 and SA002.5 are approved by MEXT (Japan). Prior to the experiments the lines had been maintained in IVF dishes on mitomycin-C inactivated mEF feeder layers in VitroHES™ medium (Vitrolife AB, Kungsbacka, Sweden, http://www.vitrolife.com) supplemented with 4 ng/ml hrbFGF and cut manually by using a micro capillary as cutting and transfer tool. To transfer hBS cells from traditional culture to the enzymatic dissociation, the used culture medium was removed and the culture dishes were washed once with PBS (Invitrogen). A volume of 0.5 mL of TrypLE™ Select (Invitrogen), Accutase™ (Chemicon) or Trypsin/EDTA (Invitrogen) was then added to each IVF dish. Dishes were incubated at 37° C. until the outer regions of the hBS cell colonies started to round up from the feeder layer. The cell sheet was then broken apart to a single cell suspension by repeated tritruration with a pipette, transferred to a centrifuge tube and centrifuged at 400 g for 5 minutes. Supernatant was discarded, the hBS cell pellet was resuspended in fresh VitroHES™ medium and the single cell suspension was seeded into an IVF dish containing a dense feeder layer of inactivated hFF.

### Example 6

### Enzymatic Propagation of hBS Cells Using Passage as Single Cell Suspension

For enzymatic propagation the hBS cells were maintained in a culture system consisting of hFF feeder cells at high density and VitroHES™ medium supplemented with 4 ng/ml hrbFGF. Enzymatic dissociation was initiated by removing the culture medium washing the culture dishes with PBS (Invitrogen). A volume of 0.5 mL of TrypLE™ Select (Invitrogen), Accutase™ (Chemicon) or Trypsin/EDTA (Invitrogen) was then added to each dish. Dishes were incubated at 37° C. until the outer regions of the hBS cell colonies started to round up from the feeder layer. The cell sheet was then broken apart to a single cell suspension by repeated tritruration with a pipette. Subsequently the cell suspension was transferred to a centrifuge tube and centrifuged at 400 g for 5 minutes. Supernatant was discarded and the hBS cell pellet was resuspended in fresh VitroHES™ medium. Cells were then seeded into fresh IVF dishes containing a dense feeder layer of inactivated hFF at split ratios between 1:4 or 1:5 and 1:500. The lower split ratio was used during what we refer to as the adjustment procedure during the very first passages in the new system. After no more than 5 passages the hBS cell line was split at split ratios between 1:20 and 1:500. Cultures were maintained in an incubator at 37° C. and 95% humidity. Used culture medium was replaced with fresh VitroHES™+4 ng/ml hrbFGF every 2-3 days. Depending on the growth speed of the individual hBS cell line the cells were passaged every 6-12 days. Both line SA001 and SA121 have when time of filing this application been cultured for more than 20 passages with maintained normal karyotype and further been frozen and thawed according to conventional slow-freezing methods in their normal culture medium supplemented with 10% DMSO.

### Example 7

Immunohistochemical and Histochemical Analysis of hBS Cells

hBS cell cultures were fixed in 4% paraformaldehyde for 15 minutes, permeabilized for 5 minutes in 0.5% trition solution (Sigma-Aldrich) and subsequently blocked with 5% FBS in PBS (Invitrogen). The cells were incubated with primary antibody solution overnight at 4° C. The primary antibodies used were specific for Oct-4, TRA-1-60, TRA-1-81, SSEA-1, SSEA-3 and SSEA-4 (Santa Cruz Biotechnology, Santa Cruz, Calif., http://www.southernbiotech.com). Incubation with FITC- or Cy3-conjugated secondary antibodies (Jackson Immunoreserach Laboratories) was performed for 60 minutes at room temperature. Cell nuclei were counterstained with DAPI (Sigma). The activity of alkaline phosphatase was determined using an alkaline phosphatase activity detection kit according to the manufacturer's instructions (Sigma-Aldrich). Stainings were evaluated and documented using a Nikon Eclipse TE-2000 U fluorescence microscope. Both SA001 and SA121 showed all the above hBS cell characteristics after more than 20 passages.

### Example 8

### Genetic Characterization

For karyotype analysis the hBS cells were incubated in the presence of colcemid, trypsinized, fixed and mounted on glass slides. The chromosomes were visualized by DAPI staining, arranged and documented using an inverted microscope equipped with appropriate filters and software (CytoVision; Applied Imaging; Santa Clara Calif., http://www.appliedimaginqcorp.com).

For fluorescence in situ hybridization (FISH) analysis, commercially available kits containing probes for chromosomes 12, 13, 17, 18, 21, X and Y were used according to the manufacturer's instruction with minor modifications. The slides were analyzed in an inverted microscope equipped with appropriate filters and software (CytoVision). SA001 and SA121 both showed normal karyotypes after being cultured in the present system for more than 20 passages. FISH was confirmed normal at even higher passages.

### Example 9

### Analysis of Pluripotency In Vivo

Pluripotency was assessed by teratoma formation in immunodeficient mice (SCID) as described earlier [Heins et al]. In brief, undifferentiated hBS cell colonies were mechanically cut into 200×200-μm pieces and surgically placed under the kidney capsule of severe combined SCID mice (C.B-17/lcrCrl-scidBR; Charles River Laboratories). The mice were sacrificed after 8 weeks and tumours were excised and fixed in 4% paraformaldehyde. Hematoxylin and eosin stained paraffin sections were evaluated histologically for the presence of differentiated human tissue derived from all three embryonic germ layers e.g., neuroectoderm, cartilage, and gut-like epithelium. All three germ layers were confirmed in the teratomas from both SA001 and SA121 cultured in the present system still after more 20 passages.

### Example 10

### Culture and Characterization of Additional hBS Cell Lines in the Enzymatic Passaging System

In addition to hBS cell lines SA001 and SA121 mentioned above, also hBS cell lines SA167 and SA002 have been successfully transferred to and cultured in the enzymatic passaging system as described above.

Characteristics of hBS cell lines cultured in the enzymatic passaging system is shown in the below table.

| | SA001 TE | SA001 TrypLE | SA121 TE | SA121 TrypLE | SA002 TrypLE | SA167 TrypLE |
|---|---|---|---|---|---|---|
| Number of | >40 | >40 | >20 | >20 | >15 | >15 |
| SCED | | | | | | |
| passages | | | | | | |
| Morphology | Undiff. | Undiff. | Undiff. | Undiff. | Undiff. | Undiff. |
| IHC | | | | | | |
| Oct-4 | + | + | + | + | + | + |
| Tra1-60 | + | + | + | + | + | + |
| Tra1-81 | + | + | + | + | + | + |
| SSEA-3 | + | + | + | + | + | + |
| SSEA-4 | + | + | + | + | + | + |
| SSEA-1 | − | − | − | − | − | − |
| ALP | + | + | + | + | + | + |
| RT-PCR | | | | | | |
| Oct-4 | + | + | ND | ND | + | + |
| Nanog | + | + | ND | ND | + | + |
| Cripto | + | + | ND | ND | + | + |
| AFP | − | − | ND | ND | − | − |
| Karyotype | 46 XY p25, p39 | 46 XYp27, p39 | 46 XYp21 | 46 XY p21 | ND | ND |
| FISH | 2n XYp25 | 2n XYp35 | 2n XY p21 | 2n XY p21 | ND | ND |
| Teratoma | Endo/Ecto/Meso | Endo/Ecto/Meso | Endo/Ecto/Meso | Endo/Ecto/Meso | ND | ND |

Abbreviations: p, number of enzymatic passages; TE, Trypsin-EDTA; TrypLE, TrypLE Select; Undiff, undifferentiated; ND, not determined; Endo, endoderm; Ecto, Ectoderm; Meso, Mesoderm.

### Example 11

### Robustness, Reproducibility and Universal Applicability of the Method

A great advantage of the invented method for enzymatic culture of hBSC lines is that it has proven to be stable, robust and easy to reproduce for several cell lines. Seven different hBSC lines have repeatedly been used for the evaluation and establishment of the method. Additionally the invented adjustment procedure for establishment of hBS cell lines to the subsequent culture method have proven to be stable and fast for several different hBS cell lines. The number of times establishment was made for each line is indicated in brackets, SA001 (6), SA002 (5), SA002.5 (>3), SA167 (3), SA348 (>4), SA461 (>10) and SA502 (2). Continuous establishment according to the invention could be made for scaled up culture of the hBS cell lines in industrial cell culture production.

### Example 12

### Colony Formation Assay

To test the supportive capacity of the enzymatic passaging and culture system, traditionally cultured hBS cells (on mEFs using mechanical passaging) were dissociated to single cells by using either TrypLE™ Select, Accutase™ or Trypsin-EDTA. The hBS cells were diluted and seeded into new IVF dishes in two densities on either hFFs or mEFs, resulting in approximately 350 and 700 hBS cells/cm². Media was changed every 2-3 days. Approximately one week later, the media was removed and the cells were washed with 1×PBS (Gibco, invitorgen) followed by alkaline phosphatase staining according to the manufacturer's description (Sigma-Aldrich). The numbers of obtained hBS cell colonies from all four test groups were counted. For semi-quantitative evaluation of the number of undifferentiated hBS cell colonies, the colonies were scored positive if more than 50% of the colony was undifferentiated when visually inspected in an inverted microscope. The experiments were performed in duplicates and repeated three times. hBS cell line SA002.5 was employed for these experiments.

Regardless of which enzyme was employed, approximately 90% of the colonies on hFFs were graded as undifferentiated (See FIG. 6). Of 200 evaluated colonies treated with Accutase™ 191 were judged as undifferentiated. For comparison, if dissociated hBS cells were plated on mEFs, significantly decreased numbers of good colonies were obtained; only around 60% of the colonies were graded as undifferentiated (FIG. 6). The qualitative differences were similar in both dilutions tested. Thus, using hFFs as feeders and TrypLE™ Select for dissociation seems to be the most favorable combination to facilitate clonal survival of undifferentiated, pluripotent hBS cells.

### Example 13

### Employing Magnetic Particles for Separation of hFF from hBS Cells

One T-75 flask with confluent hFF cells was treated with Mitomycin C for 2-3 hours in order to inhibit cell proliferation. After Mitomycin C treatment, the hFFs were washed multiple with 1×PBS where after they were dissociated to single cells by employing either 1× Trypsin-EDTA or 1× TrypLE™ Select. An aliquot of the cells were counted in a haemocytometer and the cells were diluted to appropriate concentrations. If different concentrations of magnetic particles (Endorem™) were to be tested, the cell suspensions were divided into different tubes, and different amounts of magnetic particles were then added to the tubes. The hFFs and magnetic particles were then mixed in the tube before the suspension was plated into gelatine coated IVF dishes (Falcon). Around 200,000 hFF cells were seeded per IVF dish.

Concentrations of Endorem™ tested were in a range from 5.6 ug to 560 ug per well and 200,000 cells, which corresponds to from 2.8 ug/ml to 280 ug/ml.

1-2 days after seeding a 100% medium change was performed, which removed the majority of unbound or non-endocytosed iron particles. Approximately 24 hours after the medium change hBS cells were added to the plates with hFF feeder cells.

To collect hFFs on the magnet, the cells were rinsed once in 1×PBS and dissociated with either Trypsin-EDTA or 1× TrypLE™ Select to single cells. The cell suspension was then transferred to an eppendorf tube and placed in close contact to a magnet. The cells were allowed to attach to the magnet for a few minutes and the remaining solution was then removed from the tube. After that the tube was removed from the magnet and appropriate medium or 1×PBS was added to the tube and the feeder cell containing solution was then re-suspended for cell counting and verification of separation efficiency.

At least 90% of the hFFs were caught on the magnets (90% separation efficiency) (see FIG. 7), regardless of the different concentrations tested. When enzymatically passaged hBS cells were seeded onto magnetic hFFs, the hBS cells attached, proliferated and formed cell colonies (data not shown).

### REFERENCES

Heins N, Englund M C O, Sjöblom C et al. Derivation, characterization, and differentiation of human embryonic stem cells. STEM CELLS 2004; 22:367-76.
WO03055992, A method for the establishment of a pluripotent human blastocyst-derived stem cell line, Cellartis AB
Human Embryonic Stem Cell Protocols BresaGen Inc 2004 Sjögren-Jansson E et al. Large-scale propagation of four undifferentiated embryonic stem cell lines in a feeder-free culture system. Developmental Dynamics 2005; 233:1304-1314.
Brimble et al. Karyotypic stability, genotyping, differentiation, feeder-free maintenance, and gene expression sampling in three human embryonic stem cell lines derived prior to Aug. 9, 2001. Stem Cells and Development 2004; 13:585-596
Draper J S, Smith K, Gokhale P et al., Recurrent gain of chromosomes 17q and 12 in cultured human embryonic stem cells. NAT Biotechnol 2004; 22:53-54
Buzzard J J, Gough N M, Crook J M et al. Karyotype of human ES cells during extended culture. Nat Biotechnol 2004; 22:381-382
Mitalipova, MM, Rao R R, Hoyer D M et al. Preserving the genetic integrity of human embryonic stem cells. Nat Biotechnol 2005; 23:19-20
Cowan C A et al. Derivation of embryonic stem-cell lines from human blastocysts (Special Report & Supplementary Appendix 1) New England Journal of Medicine 2004; 350; 13:1353-1356
Enver T et al, Cellular differentiation hierarchies in normal and culture-adapted human embryonic stem cells. Hum Mol Genet. 2005 Nov. 1; 14(21):3129-40
Andrews P W et al, Embryonic stem (ES) cells and embryonal carcinoma (EC) cells: opposite sides of the same coin. Biochem Soc Trans. 2005 December; 33(Pt 6):1526-30. Review

1. A culture system for propagation of human blastocyst-derived stem (hBS) cells comprising
i) human feeder cells at a density of at least 50,000 cells/cm²,
ii) one or more dissociation agents for dissociation of hBS cell colonies into a single cell suspension, and
iii) a supportive culture medium.

2. A culture system according to claim 1, wherein the density of human feeder cells is from about 50,000 to about 500,000 cells/cm².

3. A culture system according to claim 1, wherein the human feeder cells are derived from human tissue.

4. A culture system according to claim 3, wherein the human tissue is derived from embryonic, fetal, neonatal, juvenile, or adult tissue.

5. A culture system according to claim 3, wherein the human tissue is derived from skin, including foreskin, umbilical chord, muscle, lung, epithelium, placenta, fallopian tube, glandula, stroma or breast.

6. A culture system according to claim 1, wherein the human feeder cells are derived in vitro from hBS cells or cells derived from hBS cells.

7. A culture system according to claim 1, wherein the human feeder cells are derived from cell types pertaining to the group consisting of human fibroblasts, fibrocytes, myocytes, keratinocytes, endothelial cells and epithelial cells.

8. A culture system according to claim 1, wherein the human feeder cells are fibroblasts.

9. A culture system according to claim 8, wherein the feeder cells are derived from human neonatal foreskin fibroblasts.

10. A culture system according to claim 6, wherein the cells derived from hBS cells are fibroblasts or have a mesenchymal phenotype.

11. A culture system according to claim 1, wherein the human feeder cells are derived from embryonic fibroblasts, extraembryonic endoderm cells, extraembryonic mesoderm cells, fetal fibroblasts and/or fibrocytes, fetal muscle cells, fetal skin cells, fetal lung cells, fetal endothelial cells, fetal epithelial cells, umbilical chord mesenchymal cells, placental fibroblasts and/or fibrocytes, placental endothelial cells, post-natal human foreskin fibroblasts and/or fibrocytes, post-natal muscle cells, post-natal skin cells, post-natal endothelial cells, adult skin fibroblasts and/or fibrocytes, adult muscle cells, adult fallopian tube endothelial cells, adult glandular endometrial cells, adult stromal endometrial cells, adult breast cancer parenchymal cells, adult endothelial cells, adult epithelial cells or adult keratinocytes.

12. A culture system according to claim 1, wherein the human feeder cells have been growth inactivated.

13. A culture system according to claim 12, wherein the human feeder cells have been growth inactivated by mitomycin treatment.

14. A culture system according to claim 12, wherein the human feeder cells have been growth inactivated by irradiation.

15. A culture system according to claim 1, wherein the human feeder cells are seeded from about 1 to about 10 days prior to seeding the hBS cells.

16. A culture system according to claim 15, wherein the culture medium is changed at least one time prior to seeding the hBS cells.

17. A culture system according to claim 1, wherein one or more dissociation agents is an enzyme.

18. A culture system according to claim 17, wherein the enzyme is a proteolytic enzyme.

19. A culture system according to claim 17, wherein the enzyme is a collagenolytic enzyme.

20. A culture system according to claim 17, wherein the enzyme is selected from the group consisting of trypsin, trypsin-like, dispase, dispase-like, pronase, pronase-like, collagenase, collagense-like and matrix metalloproteinases.

21. A culture system according to claim 17, wherein the enzyme is a recombinant enzyme.

22. A culture system according to claim 1, wherein one or more dissociation agents is a chelating agent.

23. A culture system according to claim 22, wherein the chelating agent is a chelator of divalent cations.

24. A culture system according to claim 22, wherein the chelating agent is selected from the group consisting of EDTA, EGTA and HEDTA.

25. A culture system according to claim 1, wherein the one or more dissociation agents is a combination of at least two dissociation agents.

26. A culture system according to claim 25, wherein the combination of dissociation agents comprises one or more enzymes and one or more chelating agents.

27. A culture system according to claim 25, wherein the combination of dissociation agents is xeno-free.

28. A culture system according to claim 25, wherein the combination of dissociation agents is selected from the group of commercially available combinations consisting of TrypLE™ Select, Accutase™ and Accumax™.

29. A culture system according to claim 1, wherein the supportive culture medium is selected from the group consisting of DMEM and IMDM.

30. A culture system according to claim 1, wherein the supportive culture medium is supplemented with from about 0.5 to about 1000 ng/ml hrbFGF.

31. A culture system according to claim 1, wherein the supportive culture medium is supplemented with from about 1 to about 40% serum.

32. A culture system according to claim 31, wherein the serum is FBS or human serum.

33. A culture system according to claim 1, the culture system comprising
i) human neonatal foreskin feeder cells at a density of at least 70,000 cells/cm²,
ii) TrypLE™ Select for dissociation of hBS cell colonies into a single cell suspension, and
iii) VitroHES™ supplemented with at least 4 ng/ml hrbFGF supportive culture medium, which culture system makes it possible to propagate hBS cells by dissociation of hBS cell colonies into a single cell suspension at each consecutive passage for an extended time period, while maintaining the significant characteristics of hBS cells.

34. A method for propagation of hBS cells in a culture system as defined in claim 1, the method comprising the steps of:
i) optionally, performing an adjustment procedure in order for the hBS cells obtained from a master cell line to adjust to the culture system,
ii) dissociating the hBS cells into a single cell suspension by the use of one or more dissociation agents,
iii) distributing the single cell suspension in a split ratio of at least 1:4 into one or more culture vessels comprising human feeder cells at a density of at least 50,000 cells/cm², iv) incubating the hBS cells for from about 3 to about 25 days upon regular medium changes, and
v) repeating n times from step ii), wherein n is an integer of at least 1,
in order to propagate the hBS cells, while maintaining the significant characteristics of such cells.

35. A method according to claim 34, further comprising the steps of:
vi) analyzing the cells obtained in step iv) to see whether the significant characteristics of hBS cells are maintained, and
vii) repeating from step ii) if the significant characteristics of hBS cells are maintained.

36. A method according to claim 34, wherein n is at least 5.

37. A method according to claim 34, wherein the dissociation of hBS cell colonies into a single cell suspension in step ii) is performed by treating the hBS cell colonies with one or more dissociation agents.

38. A method according to claim 34, wherein the effect of the one or more dissociation agents is diminished prior to step iii).

39. A method according to claim 38, wherein the effect of the one or more dissociation agents is diminished by physical removal of the one or more dissociation agents, dilution of the one or more dissociation agents, addition of one or more inhibitors of the one or more dissociation agents, addition of one or more substrates of the one or more dissociation agents in excess or inherent auto-inhibition of the one or more dissociation agents.

40. A method according to claim 34, wherein the split ratio is from about 1:4 to about 1:5000.

41. A method according to claim 40, wherein the split ratio is 1:20.

42. A method according to claim 34, wherein the hBS cells obtained in step iii) are incubated for from about 3 to about 25 days.

43. A method according to claim 34, wherein the medium changes in step iv) are performed from about 1 to about 14 times a week.

44. A method according to claim 34, wherein the adjustment procedure comprises the steps of:
a) dissociating the hBS cell colonies into a single cell suspension by use of one or more dissociation agents,
b) distributing the single cell suspension in a split ratio of at least 1:3 into one or more culture vessels comprising human feeder cells at a density of at least 50,000 cells/cm²,
c) incubating the hBS cells for from about 5 to about 30 days upon medium changes at regular time intervals, and
d) optionally, repeating from step a) at the most 5 times,
in order to obtain homogeneous undifferentiated hBS cells colonies.

45. A method according to claim 44, wherein the hBS cells obtained in step c) are incubated for from about 5 to about 30 days.

46. A method according to claim 44, wherein step d) is included.

47. A method according to claim 46, wherein repetition from step a) is performed at the most 5 times.

48. A method according to claim 44, wherein the medium changes in step c) are performed from about 1 to about 14 times a week.

49. A method according to claim 34, comprising the steps of
i) performing an adjustment procedure comprising the steps of a) dissociating the hBS cell colonies into a single cell suspension by use of one or more dissociation agents, b) distributing the single cell suspension in a split ratio of at least 1:3 into one or more culture vessels comprising human feeder cells at a density of at least 50,000 cells/cm², c) incubating the hBS cells for from about 5 to about 30 days upon medium changes at regular time intervals, d) repeating from step a) at the most 5 times, in order to obtain homogeneous undifferentiated hBS cells colonies,
ii) dissociating the hBS cells into a single cell suspension by the use one or more dissociation agents,
iii) distributing the single cell suspension in a split ratio of at least 1:4 into one or more culture vessels comprising human feeder cells at a density of at least 50,000 cells/cm²,
iv) incubating the hBS cells for from about 3 to about 25 days upon regular medium changes, and
v) repeating at least 20 times from step ii),
in order to propagate the hBS cells, while maintaining the significant characteristics of such cells.

50. A single cell suspension of hBS cells, wherein less than 10% of the cells appear as clusters of cells, the single cell suspension being capable of surviving and maintaining the significant characteristics of hBS cells for more than 20 passages, when subjected to a method for propagation of such cells as defined in claim 34.

51. A xeno-free single cell suspension of hBS cells, wherein less than 10% of the cells appear as clusters of cells, the single cell suspension being capable of surviving and maintaining the significant characteristics of hBS cells for more than 20 passages, when subjected to a method for propagation of such cells as defined in claim 34.

52. An improved cell line from hBS cells, which is capable of surviving and maintaining the significant characteristics of hBS cells for more than 20 passages, when subjected to a method for propagation of such cells as defined in claim 34.

53-57. (canceled)

58. A kit comprising the following components:
i) a single hBS cell population obtainable by a method as defined in claim 34, and
ii) a user manual describing a method for propagation of the hBS cells.

59. (canceled)

60. A kit comprising, a first component comprising i) a single cell population obtainable by a method as defined in claim 34,
and at least one of the following components
ii) human feeder cells,
iii) one or more dissociation agents for dissociation of hBS cell colonies into a single cell suspension, and
iv) a supportive culture medium.

61. A kit according to claim 58, wherein the single cell population is derived from a xeno-free derived hBS cell line and any other components are xeno-free.
